(19) United States
(12) Patent Application Publication
(10) Pub. No.: US 2012/0087972 A1
(43) Pub. Date: Apr. 12, 2012

(54) Winnie wipes

(75) Inventor: Noah Ballard, Old Hickory, TN (US)

(73) Assignee: Noah Thomas Ballard, Old Hickory, TN (US)

(21) Appl. No.: 12/807,006

(22) Filed: Oct. 7, 2010

(51) Int. Cl. A61K 9/70; A61P 31/04

(52) U.S. Cl. 424/443

(57) ABSTRACT

After urinating; the Winnie Wipes would be used to remove urine from the end/tip of a Male's penis. Then, the male using his “free hand” too remove a 1 ply flexible thin plastic “back” sided wipe, from the container/dispenser; grasping the plastic “pull” “tab”. Then, dabbing/pressing the “front” napkin\tissue side; against the end/tip of his penis, causing capillary action, to force the urine out of his penis. Then, by wiping with the same side; absorbing, and containing the urine; and sanitizing; as it's prevented from contacting the male's fingers, by the flexible thin plastic urine proof sheet attached to the “back” side of the wipe. The napkin/dispenser could be made of various materials\antiseptics; that facilitate urine removal, containment and preventing urine from spreading anywhere else; and penis end/tip sanitation.

A male stands and urinates into urinals, commodes, etc; while holding/grasping his penis with one hand. And, after urinating, the male will jerk, shake, sling, or wiggle, his penis, to remove the extraneous urine from the end/tip of his penis.

Which sprays, spreads, splashes, the highly corrosive, disgusting, filthy, messy, repulsive, smelly, stainful, waste urine; upon his—arms, abdomen, chest, face, feet, fingers, groin, hands, legs, neck; his clothing—belt, jacket, pants, shirt, socks, shoes, shorts, trousers; jewelry—bracelets, rings, watches, and the nearby—commodes, partitions, urinals, ceiling, walls, and floor!

After a male urinates, the Winnie Wipes would be used too remove urine from the end/tip of his penis; by, the male using his “free hand” too remove a flexible thin plastic “back” sided wipe from the container/dispenser; by, grasping the plastic “Pull” “Tab”; then, dabbing/pressing/wiping the “front” napkin\tissue side; several times against the end/tip of his penis; which, absorbs and contains the urine as it's prevented from contacting the male's fingers\hand, by the 1 ply flexible thin plastic liquid/urine proof sheet attached To the “back” side of the wipe.

Thus, any urine that would typically create the highly corrosive, disgusting, filthy, messy, repulsive, smelly, stainful, waste urine; upon his—arms, abdomen, chest, face, feet, groin, hands, legs, neck; his clothing—belts, jackets, pants, shirts, socks, shoes, shorts, trousers; jewelry—bracelets, rings, watches, and the nearby—commodes, partitions, urinals, ceiling, walls, and floor; would be prevented from doing so!

Then, the used/soiled Winnie Wipe will be discarded in a nearby Waste Receptacle/Trash Can.

The Concept of Winnie Wipes, is to have a container\napkin\tissue\dispenser made from a 4 Ply, Napkin/Tissue (With the back side of each napkin/tissue to have a thin 1 ply flexible, plastic liquid/urine proof sheet attached, For a total of 5 plys);

That would be located next To Commodes/Urinals, etc. In the Home, or Business such As:

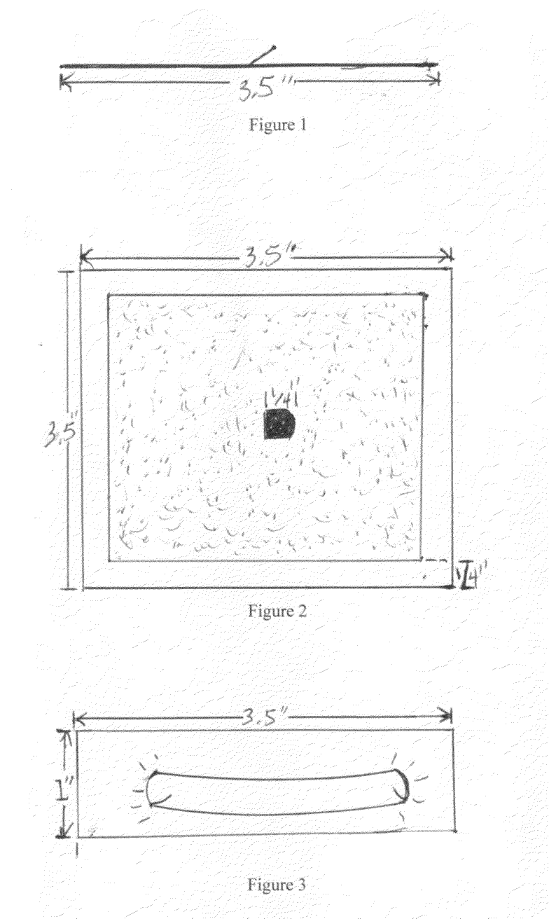

FIG. 1, Drawing below shows a side view of the 5 ply Napkin\Tissue, With a Plastic Pull Tab; which, is attached to the 5 ply napkin/tissue; which allows easy removal of a 5 ply napkin/tissue.

FIG. 1, below shows the thin 1 ply flexible, plastic liquid/urine proof sheet, with a pull tab in the up position. The pull tab would be flat underneath each Winnie Wipe 5 ply napkin/tissue.

FIG. 2, below shows the top drawing view of the 5 ply container\napkin\tissue\dispenser containing a 5 ply napkin\tissue, with a pull tab.

The FIGS. 2 and 3, below shows the Winnie Wipes container\napkin\tissue\dispenser (which could be made from Cardboard, or Plastic, or thin Sheet Metal); with a top FIG. 2, of the 5 ply container/napkin\tissue\dispenser with a plastic pull tab; which is attached to the 5 ply napkin\tissue; allowing easy removal, of a 5 ply napkin\tissue.

FIG. 2 Front\Top Drawing view as it would be seen; after it would be adhesively, or bolted, or placed upon, or fastened, or otherwise attached to a wall close to a urinal; or to the urinal; or to a commode, or toilet, etc.; or, after the Winnie Wipes, container\napkin\tissue\dispenser; could be placed on a commode top, or other conveniently accessible surface.

FIG. 2, the Winnie Wipes, napkin\tissue side; which, could be moistened with an anti bacterial liquid, that would facilitate the removal of urine, is not shown in FIG. 2, of the Winnie Wipe container/napkin\tissue\dispenser; because it is on the opposite side, of that of the thin 1 ply flexible plastic liquid\urine proof sheet; that has a pull tab.

FIG. 2, Shows the thin 1 ply flexible, plastic liquid/urine proof sheet, with aa a “dotted/marked” surface; contained inside and underneath the ¼″ border/edge of the Winnie Wipes, container\napkin\tissue\dispenser.

FIG. 3, below shows the outside drawing view, of the container\napkin\tissue\dispenser; which is “transparent” allowing the viewing of the Winnie Wipes 5 ply napkins/tissues; as they would be positioned in the Winnie Wipes, container\napkin\tissue\dispenser

FIG. 3, shows an optional smiling winnie-drawing\logo which could be placed on the outside of the Winnie Wipes, container/napkin\tissue\dispenser. Too be easily seen from the sides, or top of the Winnie Wipes, container\napkin\tissue\dispenser

1. That Winnie Wipe would be used to remove urine from the end/tip of a male's penis; by, him; dabbing, pressing, and wiping, with the “front” lightly moistened anti bacterial liquid, ½ of the napkin\tissue side; several times against the end/tip of his penis. Then, the same Winnie Wipe would be used to remove urine and anti bacterial liquid, from the end/tip of a male's penis; by, him; dabbing, pressing, and wiping, with the “front” dry, other ½ of the napkin\tissue side; several times against the end/tip of his penis. The dabbing, pressing, and wiping, would facilitate the removal of urine, both from the outside and partially from the inside of the end/tip a male's penis; and “sanitize” by the lightly moistened anti bacterial napkin\tissue side; dabbed/pressed/wiped area. The dabbing, pressing, and wiping, would facilitate the removal of urine, both from the outside and partially from the inside of the end/tip a male's penis; and “sanitize” by the lightly moistened anti bacterial napkin\tissue side; dabbed/pressed/wiped area. Then by absorbing and containing the urine as it's prevented from contacting the male's fingers\hand, by the 1 ply flexible thin plastic liquid/urine proof sheet attached to the “back” side of the wipe. And, that same napkin\tissue side; would absolutely prevent any of the highly corrosive, disgusting, filthy, messy, repulsive, smelly, stainful, extraneous, waste urine; from contacting the male's—arms, abdomen, chest, face, feet, groin, hands, legs, neck; his clothing—belts, jackets, pants, shirts, socks, shoes, shorts, trousers; jewelry—bracelets, rings, watches, and the nearby—commodes, partitions, urinals, ceiling, walls, and floor. Then, the used/soiled Winnie Wipe will be discarded in a nearby Waste Receptacle/Trash Can.
